(19) United States
(12) Patent Application Publication
(10) Pub. No.: US 2004/0254623 A1
(43) Pub. Date: Dec. 16, 2004

(54) Star suture sleeve

(75) Inventors: Luis M. Rodriguez, Coon Rapids, MN (US); Jeffrey P. Bodner, Saint Paul, MN (US); Scott M. Partridge, Blaine, MN (US)

Correspondence Address: MERCHANT & GOULD PC, P.O. BOX 2903, MINNEAPOLIS, MN, 55402-0903, US

(73) Assignee: Cardiac Pacemakers, Inc.

(21) Appl. No.: 10/461,800

(22) Filed: Jun. 12, 2003

(51) Int. Cl. A61N 1/05

(52) U.S. Cl. 607/115

(57) ABSTRACT

A tubular, suture sleeve to efficiently position and secure an implantable lead is disclosed. The sleeve comprises an outside surface and an inner lumen traversing the length of the sleeve. The outside surface comprises at least one circumferential groove adapted to receive a suture. The inner lumen comprises a star-like shaped or grooved bore. The unique shape of the lumen allows a surgeon to smoothly and easily slide the sleeve along the longitudinal axis of an implantable lead and reduces the risk of cutting through the sleeve and damaging the lead when securing the sleeve to the lead with a suture.

### TECHNICAL FIELD

The present device relates generally to a suture sleeve for an implantable medical device lead and particularly, but not by way of limitation, to such a suture sleeve that requires less force to slide the sleeve over the lead while maintaining the structural rigidity of the sleeve to prevent a suture from damaging the lead when securing the sleeve to the lead.

### BACKGROUND

Heart disease is a major health risk in the United States and elsewhere. One well-known treatment approach utilizes an implantable medical device, like a cardiac pacing device (i.e., a pacemaker) or a defibrillator, to manage a patient's heart rate or correct cardiac arrhythmias. An arrhythmia is generally defined as an abnormal cardiac rhythm.

A pacemaker delivers a relatively mild, periodic electrical impulse to epicardial or endocardial tissue as necessary to maintain normal sinus rhythm. In comparison to a pacemaker, an implanted defibrillator applies a much stronger electrical stimulus to the heart to “shock” it into a normal rhythm. The electrical charges for both implanted pacemakers and defibrillators are applied through electrically conductive leads that emanate from the medical device and terminate at an appropriate location on the tissue.

Suture sleeves are used to secure the implanted lead at the implant site. Suture sleeves are generally configured as tubular members, the cavity or lumen of which is adapted to sheathe the electrically conductive lead body of an implantable medical device. A suture sleeve also includes circumferential grooves adapted to receive a suture. The circumferential grooves facilitate wrapping the suture sleeve with a suture to secure the sleeve to the body of a lead and to a patient's body tissue, usually the fascia tissue of the heart. Suture leads are typically formed of soft, implantable elastomer material, such as silicone.

Suture sleeves come in several configurations. Some come from the implantable medical device manufacturer or other distributor with the lead already sheathed by the suture sleeve, thereby eliminating the need to thread or feed the lead through the sleeve during surgery. Other suture sleeves are separate from the lead, and the lead must be fed through the sleeve. Still other suture sleeves include a slit along the longitudinal axis of the sleeve to allow the sleeve to sheathe a lead body by passing the lead through the slit into the cavity or inner lumen of the suture sleeve.

Once the lead body is sheathed within the suture sleeve and properly positioned at the implant site, the suture sleeve is slid down the lead body to a point near the implant site and wrapped with a suture in the circumferential groove. The suture is pulled tight and tied to longitudinally secure the suture sleeve to the lead. The suture sleeve is then sutured to body tissue. Securing a suture sleeve in this manner is important to provide permanent hemostasis and lead stabilization at the implant site.

However, because suture sleeves must be moved along the longitudinal axis of the lead body during the implantation procedure and are constructed of soft, pliable material, problems may occur. For example, because the inner lumen of a typical suture sleeve is generally cylindrical, friction due to contact between the inside of the suture sleeve and the body of the lead may cause the sleeve to stick to the lead and make it difficult or impossible to slide the sleeve along the longitudinal axis of the lead body. On other occasions, and again because of the generally cylindrical configuration of most suture sleeve lumens, the surgeon or other clinician often must pull the suture tightly to secure the sleeve to the lead. If the surgeon pulls the suture too tight, the suture can cut through the soft material of the suture sleeve and the insulation surrounding the lead, thereby damaging the lead. When this happens, the lead must be replaced. Unfortunately, damage to the lead is often not detected until after the surgery is complete, thereby requiring additional surgery to correct the problem. These mishaps increase the time it takes to complete the surgical procedure and ultimately the total cost of the implantation procedure.

Thus, for these and other reasons, there is a need for a suture sleeve configured to reduce the contact areas inside the lumen of the sleeve when sliding the sleeve along the longitudinal axis of the lead body while also reducing the risk of a suture cutting through the sleeve and damaging the lead.

### SUMMARY

According to one aspect of the invention, there is provided a suture sleeve for an implantable lead comprising an elastic, tubular body further comprising an outside surface and an inner lumen. As used herein, the word lumen refers to the canal, duct or cavity defined by the tubular body of the suture sleeve. Also, as used herein, a “clinician” can be a physician, physician assistant (PA), nurse, medical technologist, or any other patient health care provider.

The outside surface may further comprise at least one circumferential groove adapted to receive a suture. In a preferred embodiment, the outside surface comprises three circumferential grooves.

The inner lumen of the suture sleeve is configured to reduce the contact areas between the sleeve and the implantable lead and reduce the force necessary to secure the sleeve to the lead. In a preferred embodiment, the inner lumen is bored in the general shape of a star, said star shape traversing the length of the tubular body.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention, which is set forth in the following claims.

### BRIEF DESCRIPTION Of The DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a perspective view, illustrating generally, among other things, an embodiment of a suture sleeve with a star-like shaped inner lumen.

FIG. 2 is a cross-section view, illustrating generally, among other things, one embodiment of an inner lumen of a suture sleeve.

FIG. 3 is a cross-section view, illustrating generally, among other things, another embodiment of an inner lumen of a suture sleeve.

### DETAILED DESCRIPTION

In the following detailed description, reference is made to the accompanying drawings that form a part hereof, and in which are shown by way of illustration specific embodiments or examples. These embodiments may be combined, other embodiments may be utilized, and structural and logical changes may be made without departing from the spirit and scope of the present invention. The following detailed description is, therefore, not to be taken in a limiting sense, and the scope of the present invention is defined by the appended claims and their equivalents.

The present device is described with respect to a suture sleeve that requires less force to slide the sleeve over a lead body and less force to suture the suture sleeve to the lead body to retain the lead in position while maintaining the sleeve's structural rigidity to prevent a suture from cutting through the sleeve.

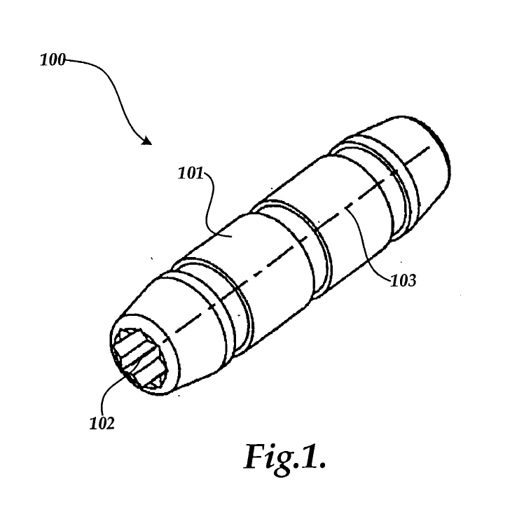

FIG. 1 is a perspective view illustrating generally, among other things, one embodiment of a tubular suture sleeve 100 comprising an outside surface 101 and an inner lumen 102. The suture sleeve 100 comprises flexible, resilient materials well known in the art. Silicone is a material commonly used in manufacturing suture sleeves. The outside surface 101 of the sleeve is adapted to accommodate a suture. The outside surface 101 may comprise at least one circumferential groove to facilitate placement of a suture. As further shown in FIG. 1, the outside surface 101 may comprise three circumferential grooves. The inner lumen 102 is adapted to reduce the frictional forces inside the sleeve 100 when sliding the sleeve along the longitudinal axis of a lead. To accomplish this, the suture sleeve 100 may be bored so that the inner lumen 102 throughout the sleeve 100 comprises a star-like shape. Alternatively, the suture sleeve 100 may be bored so that only one or more sections or lengths of the inner lumen 102 comprises a star-like shape. By either completely or partially boring the lumen 102 in this manner, the surface area of the lumen 102 coming into contact with the lead is reduced, thereby making the sleeve 100 easier to slide over the lead body. As further shown in FIG. 1, the sleeve 100 may include a slit 103 traversing a longitudinal axis of the sleeve 100, said slit adapted to receive a lead body passed there through and into the lumen 102. The elastic nature of the suture sleeve 100 biases the slit 103 to a closed position to sheathe and retain a lead body passed through the slit 103 into the inner lumen 102. With the slit 103 in the biased, closed position, the tubular body 100 along the line of the slit is essentially contiguous.

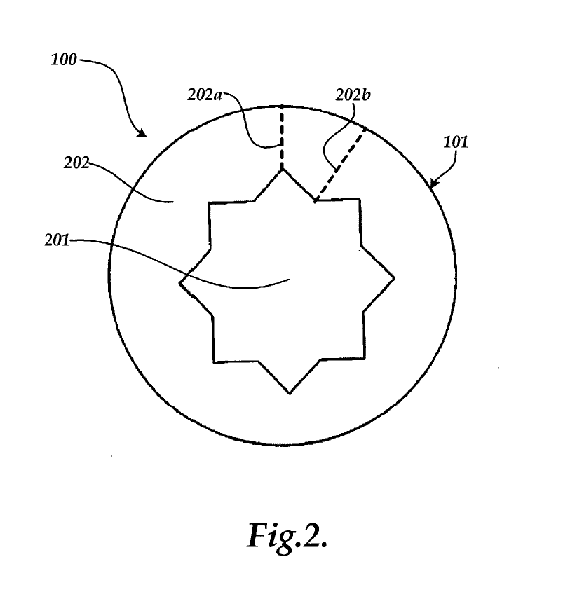

FIG. 2 is a cross-section view illustrating generally, among other things, one embodiment of a suture sleeve 100 comprising a star-like shaped bore 201. In this embodiment, the star-like bore comprises the cumulative areas of two squares, both sharing a common central axis, with one imposed upon the other and rotated about 45 degrees with respect to each other. As further shown in FIG. 2, the sleeve wall 202 relative to the outside surface 101 is thin 202a in some places and thicker 202b in other places.

It is well known in the art that the amount of force required to tighten a suture sleeve is proportional to the stiffness of the sleeve in the radial direction 202, 203. In general, stiffness increases in proportion with the thickness of a material. If the material of a suture sleeve is too thick, the sleeve will require more force to deform the sleeve to make contact with the lead body. If the suture sleeve material is too thin, the suture could cut through the sleeve wall and damage the lead. A sleeve wall 202 with a variable thickness as shown in FIG. 2 makes the sleeve resistant to cutting in some areas while also promoting deformation of the suture sleeve to contact the lead body in other areas. In addition, a star-shaped lumen 201 requires less force to tighten the suture sleeve because it conforms more easily to a lead body than a cylindrical lumen. Consequently, a clinician need not use as much force to tighten a suture around the sleeve to secure it to the lead. This reduces the risk of cutting through the sleeve and damaging the lead.

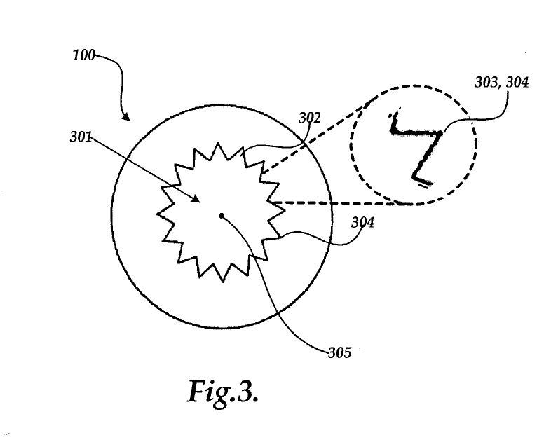

FIG. 3 is a cross-section view illustrating generally, among other things, another embodiment of a tubular, suture sleeve body 100 comprising a grooved lumen 301. Each groove 302 may traverse the full length of the tubular body or a portion or portions thereof. The grooves 302 may be equally spaced, more or less, around the circumference of the lumen 301 or arranged in a configuration that reduces frictional contact with a lead body, preserves structural rigidity and best conforms to the shape of the lead body when securing the suture sleeve to the lead. By way of non-limiting example only, the lumen 301 may comprise eighteen (18) grooves equally spaced, more or less, around the circumference of the lumen 301. As further shown in FIG. 3, and by way of further non-limiting example only, each groove may be triangular 303 with the apex 304 of the triangle distal from the radial center 305 of the lumen 301. Other embodiments of the number, pattern and spacing of the grooves may be employed to reduce frictional contact areas between the lumen and the lead to make the suture sleeve 100 easy to slide to the desired position along the lead body.

It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including,” “includes” and “in which” are used as the plain-English equivalents of the respective terms “comprising,” “comprises” and “wherein.”

What is claimed is:

1. A suture sleeve for an implantable lead body comprising:
a. a tubular body of a certain length further comprising an outside surface and an inner lumen;
b. said outside surface comprising at least one circumferential groove adapted to receive a suture; and
c. said inner lumen being defined by a sleeve wall and configured to reduce contact friction between the sleeve and the implantable lead and reduce the force necessary to secure the sleeve to the lead.

2. The suture sleeve of claim 1, wherein the tubular body further comprises a slit traversing a longitudinal axis of the tubular body and adapted to receive a lead body there through into the lumen of the tubular body.

3. The suture sleeve of claim 1, wherein the outside surface comprises a plurality of circumferential grooves.

4. The plurality of circumferential grooves of claim 3, wherein the outside surface comprises two circumferential grooves.

5. The plurality of circumferential grooves of claim 3, wherein the outside surface comprises three circumferential grooves.

6. The suture sleeve of claim 1, wherein the inner lumen is bored in a general star-like shape, said star shape traversing the full length of the tubular body.

7. The suture sleeve of claim 1, wherein the inner lumen is bored in a general star-like shape, said star shape traversing a length less than the full length of the tubular body.

8. The suture sleeve of claim 1, wherein the inner lumen is bored in a general star-like shape, said star shape traversing intermittent lengths less than the full length of the tubular body.

9. The star shape of claim 6, wherein the shape comprises the cumulative area of first and second squares sharing a common central axis, said central axis traversing the length of the tubular body, with said first and second squares areas imposed upon each other and rotated about 45 degrees with respect to each other.

10. The star shape of claim 9, wherein the shape traverses a length less than the full length of the tubular body.

11. The star shape of claim 9, wherein the shape traverses intermittent lengths less than the full length of the tubular body.

12. The suture sleeve of claim 1, wherein the inner lumen comprises grooves bored on the surface of the sleeve wall traversing the full length of the tubular body.

13. The suture sleeve of claim 1, wherein the inner lumen comprises grooves bored on the surface of the sleeve wall traversing a length less than the full length of the tubular body.

14. The suture sleeve of claim 1, wherein the inner lumen comprises grooves bored on the surface of the sleeve wall traversing intermittent lengths less than the full length of the tubular body.

15. The grooved lumen of claim 12, wherein the lumen comprises 8 grooves about equally spaced around the circumference of the lumen.

16. The grooved lumen of claim 13, wherein the lumen comprises 8 grooves about equally spaced around the circumference of the lumen.

17. The grooved lumen of claim 14, wherein the lumen comprises 8 grooves about equally spaced around the circumference of the lumen.

18. The grooved lumen of claim 12, wherein the lumen comprises 8 grooves spaced around the circumference of the lumen.

19. The grooved lumen of claim 13, wherein the lumen comprises 8 grooves spaced around the circumference of the lumen.

20. The grooved lumen of claim 14, wherein the lumen comprises 8 grooves spaced around the circumference of the lumen.

21. The grooved lumen of claim 12, wherein the lumen comprises a plurality of grooves about equally spaced around the circumference of the lumen.

22. The grooved lumen of claim 13, wherein the lumen comprises a plurality of grooves about equally spaced around the circumference of the lumen.

23. The grooved lumen of claim 14, wherein the lumen comprises a plurality of grooves about equally spaced around the circumference of the lumen.

24. The grooved lumen of claim 12, wherein the lumen comprises a plurality of grooves spaced around the circumference of the lumen.

25. The grooved lumen of claim 13, wherein the lumen comprises a plurality of grooves spaced around the circumference of the lumen.

26. The grooved lumen of claim 14, wherein the lumen comprises a plurality of grooves spaced around the circumference of the lumen.

27. The plurality of grooves of claim 21, wherein the plurality comprises 18 grooves.

28. The plurality of grooves of claim 22, wherein the plurality comprises 18 grooves.

29. The plurality of grooves of claim 23, wherein the plurality comprises 18 grooves.

30. The plurality of grooves of claim 24, wherein the plurality comprises 18 grooves.

31. The plurality of grooves of claim 25, wherein the plurality comprises 18 grooves.

32. The plurality of grooves of claim 26, wherein the plurality comprises 18 grooves.

33. The suture sleeve of claim 1, wherein the lumen comprises grooves bored on the surface of the sleeve wall traversing the full length of the tubular body, each groove comprising a triangularly-shaped groove.

34. The suture sleeve of claim 1, wherein the lumen comprises grooves bored on the surface of the sleeve wall traversing a length less than the full length of the tubular body, each groove comprising a triangularly-shaped groove.

35. The suture sleeve of claim 1, wherein the lumen comprises grooves bored on the surface of the sleeve wall traversing intermittent lengths less than the full length of the tubular body, each groove comprising a triangularly-shaped groove.

36. The grooved lumen of claim 33, wherein the lumen comprises 8 grooves about equally spaced around the circumference of the lumen.

37. The grooved lumen of claim 34, wherein the lumen comprises 8 grooves about equally spaced around the circumference of the lumen.

38. The grooved lumen of claim 35, wherein the lumen comprises 8 grooves about equally spaced around the circumference of the lumen.

39. The grooved lumen of claim 33, wherein the lumen comprises 8 grooves spaced around the circumference of the lumen.

40. The grooved lumen of claim 34, wherein the lumen comprises 8 grooves spaced around the circumference of the lumen.

41. The grooved lumen of claim 35, wherein the lumen comprises 8 grooves spaced around the circumference of the lumen.

42. The grooved lumen of claim 33, wherein the lumen comprises a plurality of grooves about equally spaced around the circumference of the lumen.

43. The grooved lumen of claim 34, wherein the lumen comprises a plurality of grooves about equally spaced around the circumference of the lumen.

44. The grooved lumen of claim 35, wherein the lumen comprises a plurality of grooves about equally spaced around the circumference of the lumen.

45. The grooved lumen of claim 33, wherein the lumen comprises a plurality of grooves spaced around the circumference of the lumen.

46. The grooved lumen of claim 34, wherein the lumen comprises a plurality of grooves spaced around the circumference of the lumen.

47. The grooved lumen of claim 35, wherein the lumen comprises a plurality of grooves spaced around the circumference of the lumen.

48. The plurality of grooves of claim 42, wherein the plurality comprises 18 grooves.

49. The plurality of grooves of claim 43, wherein the plurality comprises 18 grooves.

50. The plurality of grooves of claim 44, wherein the plurality comprises 18 grooves.

51. The plurality of grooves of claim 45, wherein the plurality comprises 18 grooves.

52. The plurality of grooves of claim 46, wherein the plurality comprises 18 grooves.

53. The plurality of grooves of claim 47, wherein the plurality comprises 18 grooves.

54. A suture sleeve for an implantable lead body comprising:
a. a tubular body further comprising an outside surface and an inner lumen;
b. said outside surface comprising three circumferential grooves, each adapted to receive a suture; and
c. a length of said inner lumen being configured in a star-like shape to reduce contact friction between the sleeve and the implantable lead, preserve structural rigidity and conform to the shape of the lead body to facilitate securing the suture sleeve to the lead.

55. The suture sleeve of claim 54, wherein the star-like shape of the inner lumen comprises an 8-pointed star.

56. The suture sleeve of claim 54, wherein the star-like shape of the inner lumen comprises a 16-pointed star.

57. The suture sleeve of claim 54, wherein the star-like shape of the inner lumen comprises an 8-pointed star, each point being about equally spaced around the circumference of the lumen.

58. The suture sleeve of claim 54, wherein the star-like shape of the inner lumen comprises a 16-pointed star, each point being about equally spaced around the circumference of the lumen.

59. A suture sleeve for an implantable lead body comprising:
a. a tubular body further comprising an outside surface and an inner lumen;
b. said tubular body further comprising a slit traversing a longitudinal axis of the tubular body and adapted to receive a lead body there through into the lumen of the tubular body;
c. said outside surface comprising three circumferential grooves, each adapted to receive a suture; and
d. a length of said inner lumen being configured in a star-like shape to reduce contact friction between the sleeve and the implantable lead, preserve structural rigidity and conform to the shape of the lead body to facilitate securing the suture sleeve to the lead, said star-like shaped lumen traversing a length of the tubular body equivalent to the length of the outside surface comprising the circumferential grooves.
